(19) United States
(12) Patent Application Publication
(10) Pub. No.: US 2010/0087700 A1
(43) Pub. Date: Apr. 8, 2010

(54) Cochlear Implant Sound Processor for Sleeping with Tinnitus Suppression and Alarm Function

(75) Inventor: Martin Zimmerling, Patsch (AT)

Correspondence Address: Sunstein Kann Murphy & Timbers LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/573,958

(22) Filed: Oct. 6, 2009

Related U.S. Application Data
(60) Provisional application No. 61/103,283, filed on Oct. 7, 2008.

(51) Int. Cl. A61F 11/04; A61N 1/36; H04R 25/00

(52) U.S. Cl. 600/25; 607/57

(57) ABSTRACT

An external processor device is described for an implanted audio prosthesis. A low profile device housing attaches on the head of a patient user over an implanted receiver coil. A limited functionality processor within the device housing generates an implant data signal consisting of special non-representational audio data not characteristic of the nearby environment. A transmitter coil within the housing in communication with the processor transmits the implant data signal to the implanted receiver coil.

This application claims priority from U.S. Provisional Patent 61/103,283, filed Oct. 7, 2008, which is incorporated herein by reference.

### FIELD OF THE INVENTION

The present invention relates to medical implants, and more specifically to a sound processor for use in cochlear implant systems.

### BACKGROUND ART

A normal ear transmits sounds as shown in FIG. 1 through the outer ear 101 to the eardrum 102, which moves the bones of the middle ear 103, which in turn excites the cochlea 104. In response to received sounds transmitted by the middle ear 103, the fluid filled cochlea 104 functions as a transducer to transmit waves to generate electric pulses that are transmitted to the cochlear nerve 113, and ultimately to the brain.

Some persons have partial or full loss of normal sensorineural hearing. Cochlear implant systems have been developed to overcome this by directly stimulating the user's cochlea 104. A typical system may include an external microphone that provides an audio signal input to an external signal processing stage 111 where various signal processing schemes can be implemented. The processed signal is then converted into a digital data format, such as a sequence of data frames, for transmission by external transmitting coil 107 into implanted processor 108. Besides extracting the audio information, the implanted processor 108 also performs additional signal processing such as error correction, pulse formation, etc., and produces a stimulation pattern (based on the extracted audio information) that is sent through connected wires 109 to an implanted electrode carrier 110. Typically, this electrode carrier 110 includes multiple electrodes on its surface that provide selective stimulation of the cochlea 104.

Existing cochlear implant systems need to deliver electrical power from outside the body through the skin to satisfy the power requirements of the implanted portion of the system. FIG. 1 shows a typical arrangement based on inductive coupling through the skin to transfer both the required electrical power and the processed audio information. As shown in FIG. 1, the external transmitter coil 107 (coupled to the external signal processor 111) is placed on the skin adjacent to the implanted processor 108. Often, a magnet in the external transmitter coil 107 interacts with a corresponding magnet in the implanted processor 108. This arrangement inductively couples a radio frequency (rf) electrical signal to the implanted processor 108, which is able to extract from the rf signal both the audio information and a power component.

In most prior systems, the external components generally have been held in separate housings so that the external transmitter coil 107 would not be in the same physical housing as the power source or the external signal processor 111. The various different physical components would generally be connected by hard wire, although some systems used wireless links between separate external components. A few systems have been proposed in which all of the external components such as an external processor and a rechargeable battery could be placed within a single housing. See U.S. Patent Publication 20080002834 (Hochmair) and U.S. Patent Publication 20070053534 (Kiratzidis), which are incorporated herein by reference.

When going to bed at night, a cochlear implant user typically turns off their external signal processor 111 and removes the external transmitter coil 107. In the morning, they perform the reverse: replacing the external transmitter coil 107 and turning back on the external signal processor 111. One problem with this routine is that the user cannot hear without the external signal processor 111, including potentially important sounds such as fire alarms and an alarm clock in the morning. In addition, some cochlear implant users experience (unpleasant) tinnitus when the external signal processor 111 is turned off and the electrical stimulation of the inner ear is interrupted.—this makes it more difficult to fall asleep.

To avoid these problems, some cochlear implant users rely on vibrating devices (pillows, wrist watches, . . . ) and/or flashing lights for alarm clocks and alarm devices. Some of those devices switch on only at predefined (programmed) times. Others which may have a built-in microphone to be able to respond if an environmental sound exceeds a certain level (e.g. in case of a fire alarm). But these attempted solutions have their own problems. For one thing, their reliability can be compromised, for example, they may fail if the user does not recognize the flashing light, of the user moves while sleeping so that their body is no longer in contact with the vibrating pillow. Moreover, such special pillows and flashing/vibrating alarm clocks are relatively large which may be a disadvantage, especially when traveling. And none of the above approaches provides any relief for tinnitus.

### SUMMARY OF THE INVENTION

An external processor device for an implanted audio prosthesis includes a low profile device housing that attaches on the head of a patient user over an implanted receiver coil. A limited functionality processor within the device housing generates an implant data signal consisting of special non-representational audio data not characteristic of the nearby environment. A transmitter coil within the housing in communication with the processor transmits the implant data signal to the implanted receiver coil. The audio prosthesis may be a cochlear implant, a middle ear implant or a bone anchored hearing aid.

In further specific embodiments, the processor may include an alarm module for detecting an alarm condition such that the implant data signal includes alarm data representing the alarm condition. For example, the alarm module may also include an alarm timer and the alarm condition may be a time-based function. In addition or alternatively, the device may also include a sensing microphone for providing an audio microphone signal to the alarm module, wherein the alarm condition is a sound-level dependent function of ambient sound detected by the sensing microphone. The processor also may include a tinnitus suppression function such that the implant data signal includes tinnitus suppression data for suppressing tinnitus in the patient user. The tinnitus suppression function may include a timer function to switch off after a predefined amount of time.

### BRIEF DESCRIPTION OF THE DRAWINGS

FIG. 1 shows elements of a typical cochlear implant system and relevant ear structures.

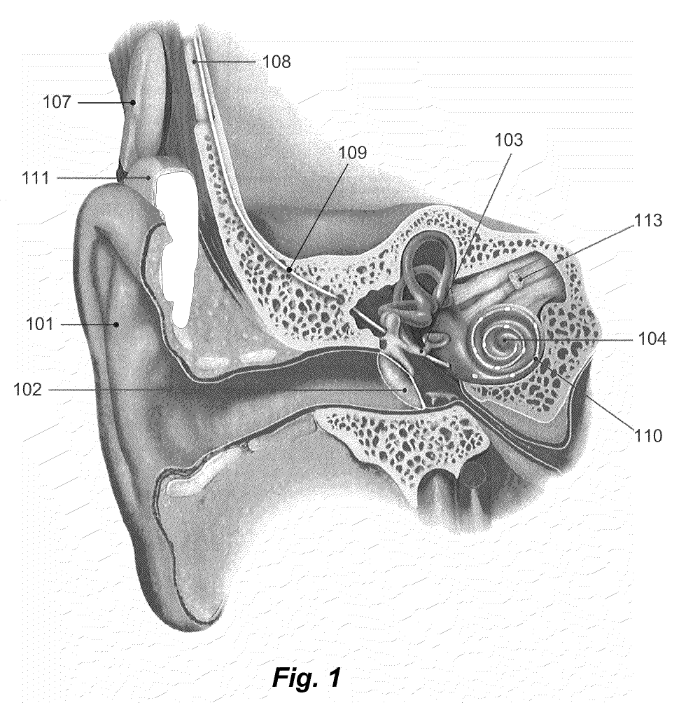

FIG. 2 shows elements of a low profile limited functionality device according to an embodiment of the present invention.

### DETAILED DESCRIPTION OF SPECIFIC EMBODIMENTS

Various embodiments of the present invention are directed to an external processor device for an implanted audio prosthesis which uses a limited functionality processor to generate an implant data signal consisting of special non-representational audio data not characteristic of the nearby environment. The resulting device is small, slim, lightweight, robust, power-saving and cheap, and can be worn by the implant user during sleep to act as an improved alarm device and/or as a tinnitus suppression device. In the following discussion, embodiments of the invention will be discussed in the specific terms of a cochlear implant system, but the invention is also broadly applicable to other types of implanted audio prostheses such as middle ear implants and bone anchored hearing aids.

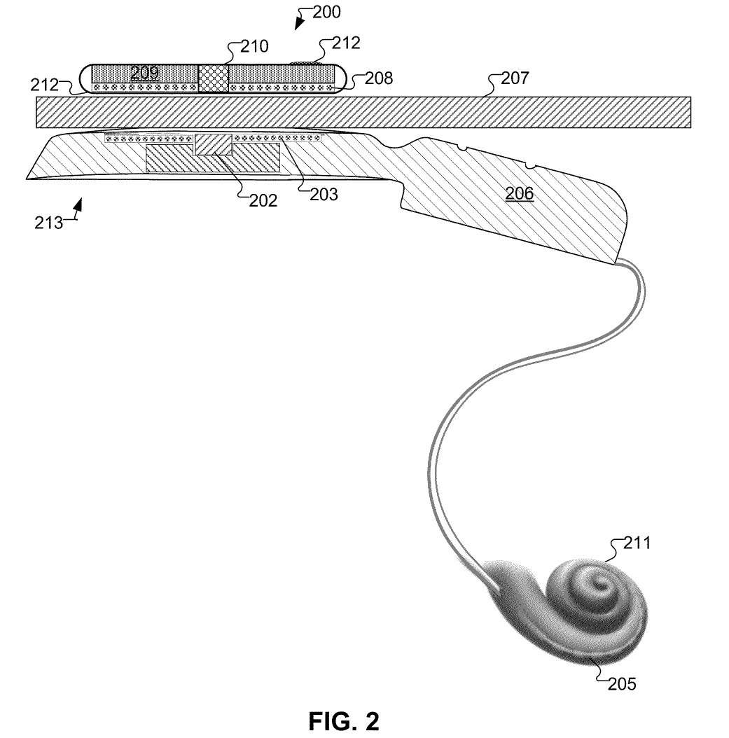

FIG. 2 shows elements of an embodiment in which a low profile device housing 200 has a generally planar skin contacting surface 212 that lies on skin of a patient user. A limited functionality processor 209 is located within the device housing 200 for developing an implant data signal consisting of special non-representational audio data not characteristic of the nearby environment (and therefore, the limited functionality processor 209 is not a speech processor). For example, the non-representational implant data signal may be a special beeping sound which corresponds to a given alarm condition. Different alarm conditions may have different associated sounds (beeps).

The processor housing 200 also contains a transmitter coil 208 in communication with the limited functionality processor 209 for coupling the implant data signal across the skin 207. The device housing 200 may also include other functionality such as a rechargeable battery arrangement that provides electrical power to the limited functionality processor 209 and the transmitter coil 208. Because of the limited functionality of the processor, the battery can be relatively cheap, small and long-lived (perhaps also in conjunction with auto/stand-by functionality of the device).

An external positioning magnet 210 is located in the radial center of the device housing 200 and magnetically interacts with a corresponding internal positioning magnet 202 to hold the external transmitter coil 208 in a fixed position on the skin 207 over an implant coil 203 in an implant housing 213 to couple the implant data signal from the transmitter coil 208 across the skin 207 to the implant coil 203. The implant coil 203 is connected to an implant processor 206 which develops a stimulation signal for the implanted electrode array 205 which stimulates audio nerve tissue in the cochlea. 211.

The entire device housing 200 is small (smaller than vibrating pillows and flashing devices), which is advantageous, particularly for traveling. In some embodiments, the magnetic holding arrangement of the external positioning magnet 210 and the internal positioning magnet 202 may be supplemented by other means such as a snood-type cap (hair net), a tape, clip, etc., and the device housing 200 reliably holds in place even when the cochlear implant user turns over during sleep.

In further specific embodiments, the limited functionality processor 209 may include an alarm module for detecting an alarm condition such that the implant data signal includes alarm data representing the alarm condition. For example, the alarm module may also include an alarm timer and the alarm condition may be a time-based function to act as an alarm clock. In some specific embodiments, the alarm module may be implanted as a hardware device or a computer software module for the limited functionality processor 209. In addition or alternatively, the device housing 200 may also include a sensing microphone 212 for providing an audio microphone signal to the alarm module so that the alarm condition is a sound-level dependent function of ambient sound detected by the sensing microphone 212. Alternatively, in some embodiments a limited functionality processor may be implemented as a function in a conventional speech processor for an audio implant system rather than as a separate device. In either case, though, a low-profile device housing 200 is preferable. In contrast to other alarm devices, the limited functionality processor 209 generates a “private alarm” which is only for the cochlear implant user and does not affect other persons sleeping in the same room.

The limited functionality processor 209 also may include a tinnitus suppression module (implemented as a hardware device and or a computer software module) in which case, the implant data signal will include tinnitus suppression data for suppressing tinnitus in the patient user. In some embodiments, the tinnitus suppression module may include a timer function to switch off after a predefined amount of time to save battery power. In another embodiment, the tinnitus suppression module may include a timer function which can be individually adjusted to a patient's need for suppressing their tinnitus.

Some aspects of various embodiments of the invention may be implemented in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments can be implemented as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. An external processor device for an implanted audio prosthesis, the device comprising:
a low profile device housing for attachment on the head of a patient user over an implanted receiver coil;
a limited functionality processor within the device housing for generating an implant data signal consisting of special non-representational audio data not characteristic of the nearby environment; and
a transmitter coil within the housing in communication with the processor for transmitting the implant data signal to the implanted receiver coil.

2. A device according to claim 1, wherein the processor includes an alarm module for detecting an alarm condition such that the implant data signal includes alarm data representing the alarm condition.

3. A device according to claim 2, wherein the alarm module includes an alarm timer and the alarm condition is a time-based function.

4. A device according to claim 2, wherein the device further comprises:
a sensing microphone for providing an audio microphone signal to the alarm module, wherein the alarm condition is a sound-level dependent function of ambient sound detected by the sensing microphone.

5. A device according to claim 1, wherein the processor includes a tinnitus suppression function such that the implant data signal includes tinnitus suppression data for suppressing tinnitus in the patient user.

6. A device according to claim 5, wherein the tinnitus suppression function includes a timer function to switch off after a predefined amount of time.

7. A device according to claim 1, wherein the implanted audio prosthesis is a cochlear implant.

8. A device according to claim 1, wherein the implanted audio prosthesis is a middle ear implant.

9. A device according to claim 1, wherein the implanted audio prosthesis is a bone anchored hearing aid.

10. A method of producing a data signal for an implanted audio prosthesis, the method comprising:
generating an implant data signal for the implanted audio prosthesis consisting of special non-representational audio data not characteristic of the nearby environment; and
transmitting the implant data signal to a implanted receiver coil of the implanted audio prosthesis.

11. A method according to claim 10, further comprising:
detecting an alarm condition; and
including alarm data representing the alarm condition in the implant data signal.

12. A method according to claim 11, wherein the alarm condition is a time-based function.

13. A method according to claim 11, wherein the alarm condition is a sound-level dependent function of ambient sound detected by a sound sensing microphone.

14. A method according to claim 10, wherein the implant data signal includes tinnitus suppression data for suppressing tinnitus in the patient user.

15. A method according to claim 14, wherein the tinnitus suppression function is a time-based function that switches off after a predefined amount of time.

16. A method according to claim 11, wherein the implanted audio prosthesis is a cochlear implant.

17. A method according to claim 11, wherein the implanted audio prosthesis is a middle ear implant.

18. A method according to claim 11, wherein the implanted audio prosthesis is a bone anchored hearing aid.

19. A computer program product in a computer readable storage medium, the product including program code for producing a data signal for an implanted audio prosthesis, the product comprising:
program code for generating an implant data signal for the implanted audio prosthesis consisting of special non-representational audio data not characteristic of the nearby environment; and
program code for transmitting the implant data signal to a implanted receiver coil of the implanted audio prosthesis.

20. A product according to claim 19, further comprising:
program code for detecting an alarm condition; and
program code for including alarm data representing the alarm condition in the implant data signal.

21. A product according to claim 20, wherein the alarm condition is a time-based function.

22. A product according to claim 20, wherein the alarm condition is a sound-level dependent function of ambient sound detected by a sound sensing microphone.

23. A product according to claim 19, wherein the implant data signal includes tinnitus suppression data for suppressing tinnitus in the patient user.

24. A product according to claim 23, wherein the tinnitus suppression function is a time-based function that switches off after a predefined amount of time.

25. A product according to claim 19, wherein the implanted audio prosthesis is a cochlear implant.

26. A product according to claim 19, wherein the implanted audio prosthesis is a middle ear implant.

27. A product according to claim 19, wherein the implanted audio prosthesis is a bone anchored hearing aid.
